(19) United States
(12) Patent Application Publication
(10) Pub. No.: US 2005/0196463 A1
(43) Pub. Date: Sep. 8, 2005

(54) NO-containing complexes

(76) Inventors: James Alexander Robert Stiles, Toronto (CA); David James Field, Edmonton (CA)

Correspondence Address: FISH & RICHARDSON PC, 225 FRANKLIN ST, BOSTON, MA, 02110, US

(21) Appl. No.: 11/070,903

(22) Filed: Mar. 3, 2005

Related U.S. Application Data
(60) Provisional application No. 60/549,656, filed on Mar. 3, 2004.

(51) Int. Cl. A61K 33/38

(52) U.S. Cl. 424/618

(57) ABSTRACT

NO-containing complexes, as well as methods of making and using such complexes are disclosed.

### CROSS-REFERENCE TO RELATED APPLICATION

This application claims priority under 35 U.S.C. §119(e)(1) to U.S. Ser. No. 60/549,656, filed Mar. 3, 2004, and entitled “NO-Containing Complexes”, which is herby incorporated by reference.

### TECHNICAL FIELD

The invention generally relates to NO-containing complexes, as well as methods of making and using such complexes.

### BACKGROUND

Nitric oxide (NO) is a biologically significant molecule.

### SUMMARY

The invention generally relates to NO-containing complexes, as well as methods of making and using such complexes. In general, a NO-containing complex can be formed by a process that includes reacting a metal-containing species, such as a nanocrystalline silver-containing material. A NO-containing complex can be used, for example, to treat a subject having a NO-related condition, and/or to prophylactically treat a subject for a NO-related condition.

In one aspect, the invention features a method of treating a subject having a NO-related condition. The method includes recognizing that the subject has the NO-related condition, and administering to the subject a therapeutically effective amount of a nanocrystalline metal-containing material.

In another aspect, the invention features a method of treating a subject having a NO-related condition. The method includes recognizing that the subject has a NO-related condition, and administering to the subject a therapeutically effective amount of a source of solution phase neutral silver.

In a further aspect, the invention features a method of treating a subject having a NO-related condition. The method includes recognizing that the subject has a NO-related condition, and administering to the subject a therapeutically effective amount of a NO-containing complex.

In an additional aspect, the invention features a method of treating a subject susceptible to having a NO-related condition. The method includes recognizing that the subject is susceptible to having a NO-related condition, and administering to the subject a therapeutically effective amount of a nanocrystalline metal-containing material.

In one aspect, the invention features a method of treating a subject susceptible to having a NO-related condition. The method includes recognizing that the subject is susceptible to having the NO-related condition, and administering to the subject a therapeutically effective amount of a source of solution phase neutral silver.

In another aspect, the invention features a method of treating a subject susceptible to having a NO-related condition. The method includes recognizing that the subject is susceptible to having the NO-related condition, and administering to the subject a therapeutically effective amount of a NO-containing complex.

In another aspect, the invention features a method that includes combining a nanocrystalline metal-containing material with a solution including a nitrogen-containing species to form a NO-complex.

In a further aspect, the invention features a method of treating a subject having a NO-related condition. The method includes recognizing that the subject has the NO-related condition, and administering to the subject a therapeutically effective amount of a metal-containing compound capable of producing NO.

In an additional aspect, the invention features a method of treating a subject susceptible to having a NO-related condition. The method includes recognizing that the subject is susceptible to having the NO-related condition, and administering to the subject a therapeutically effective amount of a metal-containing compound capable of producing NO.

In one aspect, the invention features a method that includes combining a compound with a solution including a nitrogen-containing species to form a NO-complex. The compound is capable of producing NO.

In another aspect, the invention features a method that includes combining a compound with a solution including a nitrogen-containing species to form a NO-complex. The compound is capable of producing neutral silver in the solution.

In a further aspect, the invention features a method that includes exposing a nanocrystalline metal-containing material to a nitrogen-containing gas to increase an amount of nitrogen contained in the nanocrystalline metal-containing material.

In an additional aspect, the invention features a method of treating a subject having a NO-related condition. The method includes recognizing that the subject has the NO-related condition, and administering to the subject a therapeutically effective amount of an atomically disordered, crystalline metal-containing material.

In one aspect, the invention features a method of treating a subject susceptible to having a NO-related condition. The method includes recognizing that the subject is susceptible to having a NO-related condition, and administering to the subject a therapeutically effective amount of an atomically disordered, crystalline metal-containing material.

In another aspect, the invention features a method that includes combining an atomically disordered, crystalline metal-containing material with a solution including a nitrogen-containing species to form a NO-complex.

In a further aspect, the invention features a method that includes exposing an atomically disordered, crystalline metal-containing material to a nitrogen-containing gas to increase an amount of nitrogen contained in the nanocrystalline metal-containing material.

Embodiments can feature one or more of the following aspects.

The metal-containing material can include a silver-containing material.

A nanocrystalline silver-containing material can be an atomically disordered, crystalline material.

The metal-containing material can form a NO-containing complex.

Prior to being administered to the subject, the metal-containing material can be in contact with a solution (e.g., an aqueous solution, such as saline or a body fluid). The metal-containing material can be injected into the subject, topically applied to the subject, ingested by the subject and/or inhaled by the subject.

The metal-containing material can be, for example, administered as a powder. method nanocrystalline metal-containing material can be, for example, administered in the form of a tape, a pill, a capsule, a tablet, a lozenge or a suppository. The nanocrystalline metal-containing material can be, for example, administered in the form of a pharmaceutical composition that includes the nanocrystalline metal-containing material and at least one pharmaceutically acceptable carrier.

Other features and advantages of the invention will be apparent from the description and drawings.

### BRIEF DESCRIPTION OF THE FIGURES

FIG. 1. is a schematic view of a deposition system.

FIG. 2 is an optical absorption spectrum.

FIG. 3 is an optical absorption spectrum.

FIG. 4 is an optical absorption spectrum for Silcryst®-coated Acticoat® dressing dissolved in nitric acid.

FIG. 5 shows the silver concentration and optical absorption spectrum (371 nanometers) for Silcryst®-coated Acticoat® dressing dissolved in nitric acid.

FIG. 6 shows optical absorption spectra for 3000 parts per million of silver dissolved from Silcryst®-coated Acticoat® dressing in 39% nitric acid.

FIG. 7 is an optical absorption spectrum of a solution of metallic copper in nitric oxide.

FIG. 8 is an optical absorption spectrum for nitric acid in equilibrium with an atmosphere of 10% nitric oxide in nitrogen FIG. 9 shows the optical absorbance at 372 nanometer of solutions of nitric oxide in nitric acid as a function of nitric oxide concentration.

FIG. 10 shows optical absorption spectra for 0.8M nitric oxide equilibrated with an atmosphere of 10% nitric oxide in nitrogen and then pH adjusted using sodium hydroxide.

FIG. 11 shows optical absorption spectra for solutions of Silcryst®-coated Acticoat® dressing in nitric acid that are adjusted to the indicated pH.

FIG. 12 shows optical absorbance at 370 nanometer for dilutions of a solution of Silcryst®-coated Acticoat® dressing in nitric acid.

FIG. 13 shows an optical absorbance spectrum for species removed in 100 Dalton dialysis for Silcryst®-coated Acticoat® dressing dissolved in HNO₃. The sample cell contains undialyzed solution and the reference cell contains dialyzed solution.

FIG. 14 shows an optical absorbance spectrum for a solution of Silcryst®-coated Acticoat® dressing dissolved in 0.08M nitric acid adjusted to a pH of 3.4.

FIG. 15 shows a comparison of the optical absorption spectra for dialyzed and undialyzed solutions of Silcryst®-coated Acticoat® dressing dissolved in reverse osmosis water. The absorbance for the dialyzed solution is multiplied by a factor of 4. Also shown is the spectrum for Silcryst®-coated Acticoat® dressing dissolved in saline solution.

FIG. 16 shows an optical absorption spectrum for Silcryst®-coated Acticoat® dressing dissolved in reverse osmosis water at 37° C. for 336 hours.

FIG. 17 shows optical absorption spectra for retained species in a 1000 Dalton dialysis experiment.

FIG. 18 shows optical absorption spectra for Silcryst®-coated Acticoat® dressing dissolution in 0.9% saline solution for 50 hours.

FIG. 19 shows optical absorption spectra for Silcryst®-coated Acticoat® dressing dissolution in 0.9% Saline solution for 384 hours.

### DETAILED DESCRIPTION

Preparation of NO-Containing Complexes

Without wishing to be bound by theory, it is believed that a NO-containing complex can be formed by a process that includes reacting a nanocrystalline silver-containing material with a nitrogen-containing species (e.g., nitrate ions) to provide solution phase neutral silver, and reacting the neutral phase silver as follows:
Ag+2H⁺+2NO₃⁻⁻→Ag⁺+NO₃⁻⁻+NO+2OH⁻⁻
The resulting NO can complex with silver (e.g., silver clusters, ionic silver) present in the solution to form a NO-containing complex (a complex in which NO is bound, either physically or chemically, to another species, such as a cluster or an ion). It is believed that the solution phase NO-containing complex can be in equilibrium or near-equilibrium with solution phase unbound NO. It is also believed that, provided sufficient nanocrystalline silver-containing material and nitrogen-containing species are present, as unbound silver is used, more unbound silver is formed so that a steady state concentration or a concentration within an acceptable therapeutic range of unbound NO can be provided.
Nanocrystalline Silver-Containing Materials

As referred to herein, a nanocrystalline silver-containing material is a single-phase polycrystal or a multi-phase polycrystal having a maximum dimension of about 100 nanometers or less (e.g., about 90 nanometers or less, about 80 nanometers or less, about 70 nanometers or less, about 60 nanometers or less, about 50 nanometers or less, about 40 nanometers or less, about 30 nanometers or less, about 25 nanometers or less) in at least one dimension.

In certain embodiments, a nanocrystalline silver-containing material, when contacted with an alcohol or water-based electrolyte, releases silver into the alcohol or water-based electrolyte (e.g., as ions, atoms, molecules and/or clusters) over a time scale of at least about one hour (e.g., at least about two hours, at least about 10 hours, at least about a day). Examples of alcohols and/or water-based electrolytes include body fluids and body tissue (e.g., skin, muscle, bone).

In some embodiments, a nanocrystalline silver-containing material can be an atomically disordered, crystalline silver-containing material. As referred to herein, an atomically disordered, crystalline silver-containing material means a material that has more long range ordered, crystalline structure (a lesser degree of defects) than the material has in a fully amorphous state, but that also has less long range, ordered crystalline structure (a higher degree of defects) than the material has in a bulk crystalline state, such as in the form of a cast, wrought or plated material. Examples of defects include point defects, vacancies, line defects, grain boundaries, subgrain boundaries and amorphous regions. Point defects are defects on a size scale of no more than about four atomic spacings. A vacancy is the omission of an atom from its regular atomic site in the crystal lattice. Line defects are defective regions (e.g., edge dislocations, screw dislocations) that result in lattice distortions along a line (which may or may not be a straight line), and generally have a longer scale than point defects. In an edge dislocation, a lattice displacement is produced by a plane of atoms that forms a terminus of the lattice. In a screw dislocation, part of the lattice is displaced with respect to an adjacent part of the lattice. Grain boundaries separate regions having different crystallographic orientation or misorientation (e.g., high angle grain boundaries, low angle grain boundaries, including tilt boundaries and twist boundaries). Subgrain boundaries refer to low angle grain boundaries. An amorphous region is a region that does not exhibit long range, ordered crystalline structure. In certain embodiments, an atomically disordered, crystalline silver-containing material has a degree of atomic disorder that is about the same as the degree of atomic disorder of the nanocrystalline silver coating of a member of the Silcryst®-coated Acticoat® family of dressings Smith & Nephew, Hull, UK) (e.g., a Silcryst®-coated Acticoat® dressing, a Silcryst®-coated Acticoat® dressing, a Silcryst®-coated Acticoat® moisture coating dressing, a Silcryst®-coated Acticoat® absorbent dressing). In some embodiments, an atomically disordered, crystalline silver-containing material has a degree of atomic disorder that is about the same as the degree of atomic disorder of the nanocrystalline silver coatings having a CZOI of at least five millimeters that are disclosed in the examples of Burrell et al., U.S. Pat. No. 5,958,440.

The nanocrystalline silver-containing material can be provided in a variety of forms. As an example, the nanocrystalline silver-containing material can be in the form of a powder (e.g., by dry powder inhalation, by ingestion, by needleless injection, by sprinkling on a desired area of the subject). In such embodiments, the aqueous solution can be body fluid.

As another example, a powder of nanocrystalline silver-containing material can be added to an aqueous solution and/or the powder can be disposed in a container (e.g., a tea bag-type container) that is immersed in the aqueous solution. The solution can be injected (e.g., with a needle, with a needleless injector) into the subject. The solution can be formed into an aerosol (e.g., using a mechanical mister, such as a spray bottle or a nebulizer), and the aerosol can be administered to the subject using an appropriate device (e.g., a hand held inhaler, a mechanical mister, a spray bottle, a nebulizer, an oxygen tent). The solution can be administered to the subject using an IV. The subject can swallow and/or gargle the solution. The solution can be sprayed onto a desired area of the subject. The solution can be administered to the subject by an enema.

As further example, the nanocrystalline silver-containing material can be in the form of an article including a coating of the nanocrystalline silver-containing material on a substrate (e.g., a dressing). Examples include the Silcryst®-coated Acticoat® family of dressings noted above. In such embodiments, the article can be disposed in the aqueous solution. In such embodiments, the aqueous solution can be a body fluid. In some embodiments, the nanocrystalline material may also be in the form of atomic clusters in solution or suspended in an aqueous or protic solvent (e.g., in the form of a dispersion or a suspension, such as a nanosupension). In certain embodiments, the nanocrystalline material may be an article containing the nanocrystalline silver-containing material in the form of, such as, for example, a tape, a pill, a capsule, a tablet, a lozenge or a suppository. Such an article can be partially or entirely formed of the nanocrystalline silver-containing material. Optionally, the article can be a sustained release article (e.g., a sustained release capsule) which can allow the silver-containing material to be released at a predetermined rate (e.g., a relatively constant rate) In some embodiments, the article can include a material (e.g., in the form of a coating and/or in the form of a matrix material) that allows the article to pass through certain portions of the gastrointestinal system with relatively little (e.g., no) release of the silver-containing material, but that allows a relatively large amount of the silver-containing material to be released in a desired portion of the gastrointestinal system. As an example, the article can be an enteric article (e.g., an enteric coated tablet) so that the article to passes through the stomach with little (e.g., no) silver-containing material being released, and so that the silver-containing material is relatively easily released by the article in the intestines. The article can be orally administered to the subject (e.g., by placing the article in the subject's oral cavity).

In some embodiments, the nanocrystalline material can be disposed in a pharmaceutical composition including one or more pharmaceutically acceptable carriers (e.g., creams, ointments, gels, lotions, pastes, foams, liposomes) to form, for example, a semi-solid, a water-based hydrocolloid, an oil-in-water emulsion, a water-in-oil emulsion, a non-dried gel, and/or a dried gel. The pharmaceutical composition can be, for example, topically applied to the subject.

Combinations of forms of nanocrystalline silver-containing material and/or methods of administering nanocrystalline silver-containing material can be used.

In general, a coating of nanocrystalline silver-containing material can be formed on a substrate using a desired technique. In certain embodiments, the coating is formed by depositing the material on the substrate surface using chemical vapor deposition, physical vapor deposition, and/or liquid phase deposition. Exemplary deposition methods include vacuum evaporation deposition, arc evaporation deposition, sputter deposition, magnetron sputter deposition and ion plating.

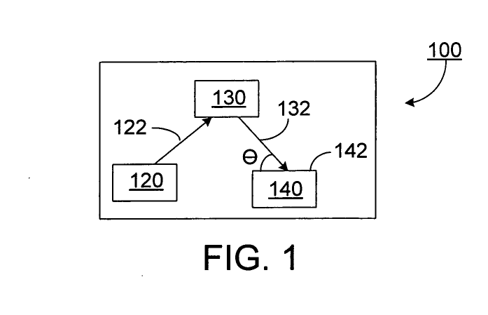

In some embodiments, the coating is prepared using physical vapor deposition. FIG. 1 shows a vapor deposition system 100 that includes a vacuum chamber 110, an energy source 120 (e.g., an electron beam source, an ion source, a laser beam, a magnetron source), a target 130 and a substrate 140. During operation, energy source 120 directs a beam of energy 122 to target 130, causing material 132 to be removed (e.g., by evaporation) from target 130 and directed to a surface 142 of substrate 140. At least a portion of the removed material 132 is deposited on surface 142.

In general, the values of the system parameters (e.g., the temperature of surface 142, the pressure of chamber 110, the gas composition within chamber 110, the angle of incidence of removed material 132 on surface 142, the distance between target 130 and surface 142) can be selected as desired. The temperature of surface 142 can be relatively low during the deposition process. For example, during the deposition process, the ratio of the temperature of substrate 140 to the melting point of the material forming target 130 (as determined in using Kelvin) can be about 0.5 or less (e.g., about 0.4 or less, about 0.35 or less, about 0.3 or less).

The angle of incidence of removed material 132 on surface 142 (θ) can be relatively low. For example, the angle of incidence of removed material 132 on surface 142 can be about 75° or less (e.g., about 60° or less, about 45° or less, about 30° or less). The distance between target 130 and surface 142 can be selected based upon the values of the other system parameters. For example, the distance between target 130 and surface 142 can be about 250 millimeters or less (e.g., about 150 millimeters or less, 125 millimeters or less, about 100 millimeters or less, about 90 millimeters or less, about 80 millimeters or less, about 70 millimeters or less, about 60 millimeters or less, about 50 millimeters or less, about 40 millimeters or less).

The pressure in chamber 110 can be relatively high. For example, vacuum evaporation deposition, electron beam deposition or arc evaporation, the pressure can be about 0.01 milliTorr or greater. For gas scattering evaporation (pressure plating) or reactive arc evaporation, the pressure in chamber 110 can be about 20 milliTorr or greater. For sputter deposition, the pressure in chamber 110 can be about 75 milliTorr or greater. For magnetron sputter deposition, the pressure in chamber 110 can be about 10 milliTorr or greater. For ion plating, the pressure in chamber 110 can be 200 milliTorr or greater.

In some embodiments, a nitrogen-containing gas (e.g., gaseous molecular nitrogen, atomic nitrogen generated from a plasma, ionized nitrogen generated from a plasma, electronically excited gaseous molecular nitrogen, metastable gaseous molecular nitrogen, gaseous NO, other molecular gases having atomic or molecular nitrogen as a component) can be present in (e.g., deliberately introduced into) chamber 110 as the silver is being deposited on the substrate. Without wishing to be bound by theory, it is believed that increasing the amount of gaseous nitrogen-containing species in chamber 110 can result in a nanocrystalline silver-containing material with a relatively large amount of nitrogen. In some embodiments, the partial pressure of gaseous nitrogen-containing species in chamber 110 during formation of the nanocrystalline silver-containing material can be at least about 0.005 milliTorr (e.g., at least about 0.01 volume percent of the sputtering gas at a total pressure of 40 milliTorr) and/or at most about five milliTorr (e.g., 10 volume percent at a total pressure of 50 milliTorr). Optionally, higher pressures of nitrogen-containing species can be used provided that they do not adversely affect the physical structure or stability of the nanocrystalline silver-containing material. In certain embodiments, the nanocrystalline-silver containing material can contain at least about 0.01 weight percent nitrogen (e.g., at least about 0.05 weight percent nitrogen, at least about 0.1 weight percent nitrogen), and/or at most about 11.5 weight percent nitrogen (e.g., at most about 10 weight percent nitrogen, at most about five weight percent nitrogen, at most about two weight percent nitrogen, at most about one weight percent nitrogen).

While FIG. 1 shows one embodiment of a deposition system, other embodiments are possible. For example, the deposition system can be designed such that during operation the substrate moves along rollers. Additionally or alternatively, the deposition system may contain multiple energy sources, multiple targets, and/or multiple substrates. The multiple energy sources, targets and/or substrates can be, for example, positioned in a line, can be staggered, or can be in an array.

A powder of nanocrystalline-silver containing material can be prepared by, for example, cold working or compressing to impart atomic disorder to the powder. In certain embodiments, a powder is prepared by forming a coating of the material as described above, and then removing the material from the surface of the substrate. For example, the material can be scraped from the surface of the substrate by one or more scrapers. In embodiments in which the substrate moves during deposition of the material, the scrapers can remove the material as the substrate moves. The scrapers can be, for example, suspended above the substrate. Such scrapers can be, for example, weighted and/or spring loaded to apply pressure sufficient to remove the material as the substrate moves. In some embodiments (e.g., when a continuous belt is used), the scrapers can be located above the end rollers to remove the material with a reverse dragging action as the substrate rounds the end roller.

Nitrogen-Containing Species

In some embodiments, the nitrogen-containing species can be supplied to the aqueous solution separately from the nanocrystalline silver-containing material. As an example, the nitrogen-containing species can be supplied to the solution in the form of ions (e.g., nitrate ions, nitrite ions).

In certain embodiments, the nanocrystalline silver-containing material can be the source of the nitrogen-containing species. As an example, the nitrogen-containing species can be provided by the reaction of reducible nitrogen present in the nanocrystalline silver-containing material with species already present in the solution.

Combinations of nitrogen-containing species can be used.

In some embodiments, the nitrogen-containing species is separately supplied to the solution. As an example, the nitrogen-containing species can be in the form of ions (e.g., nitrate ions, nitrite ions).

In certain embodiments, the nitrogen-containing species is present as part of an atomic or molecular cluster that is contiguous with or simultaneously introduced into the solution with the nanocrystalline silver-containing material in the solution. As an example, the nitrogen-containing species can be in the form of reducible nitrogen present in the nanocrystalline silver-containing material (e.g., in the form of nitrogen atoms present within the crystal lattice of the nanocrystalline silver-containing material, in the form of nitrogen-containing compounds intimately mixed with the nanocrystalline silver-containing material).

The Aqueous Solution

In some embodiments, prior to addition of the nanocrystalline silver-containing material and the nitrogen-containing species, the aqueous solution contains one or more species in addition to water. As an example, the aqueous solution can be a saline solution or sterile water. As another example, the aqueous solution can be or a body fluid. Examples of body fluids include blood, sweat, saliva, blood plasma, gastro-intestinal fluids, ocular fluids, reproductive fluids, synovial fluids, intracellular fluids, lacrimal fluids, lymphatic fluids and spinal fluids.

Use of NO-Containing Complexes

The NO-containing complexes can be used to treat a subject having one or more NO-related conditions, and/or the NO-containing complexes can be used to prophylactically treat a subject for one or more NO-related conditions.

In general, a subject can be a human or an animal (e.g., a dog, a cat, a horse, a bird, a reptile, an amphibian, a fish, a turtle, a guinea pig, a hamster, a rodent, a cow, a pig, a goat, a primate, a monkey, a chicken, a turkey, a buffalo, an ostrich, a sheep, a llama).

A “NO-related condition” refers to a condition that is treated by the production, creation and/or use of NO. Examples of NO-related conditions include immunity, analgesia, vascular disorders, neurological disturbances and degeneration, bacterial infections, fungal infections, yeast infections, viral infections, tumors, apoptotsis modulation, genetic damage, inflammation, pulmonary, hypertension and impotence. Examples of NO-related conditions include hypertension, erectile dysfunction and angina pectoris. NO-related conditions are disclosed, for example, in N. Toda et al., Pharmacol Rev Vol. 55, pp. 271-324 (2003); S. Moncada et al., Pharmacol Rev Vol. 43, pp. 109-142 (1991); D. Stuehr, Annual Review of Pharmacology and Toxicology Vol. 37, pp. 339-359 (1997); S. Lamas et al, Meth. Find. Exp. Clin. Pharmacol., Vol. 19 (supp A), pp. 13-15 (1997); L. Rivas-Cabanero et al., Exp. Nephrol. Vol. 5, pp. 23-30 (1997); D. Perez-Sala et al., Biochem. Biophys. Res. Commun., Vol. 241, pp. 7-12 (1997); P. Hernandez et al., Nefrologia XVIII, Supp. 1, 100-110 (1998); P. Hernandez et al., J. Clin. Investigation, Vol. 101, pp. 2711-2719 (1998); S. Lamas et al., Trends in Pharmacological Sciences (TIPS) Vol. 19, pp. 436-438 (1998); S. Lopez-Ongil, British Journal of Pharmacology Vol. 124, pp. 447-454 (1998); A. Navarro et al., Nefrologia XVIII, Supp. 1, pp. 79-83 (1998); W. H. Koppenol, Free Radical Biology and Medicine, Vol. 25, Nos. 4/5, pp. 385-391 (1998); L. Nikfarjam et al, Iranian J. Med. Sci., Vol. 24, Nos. 3/4, pp. 135-173, (1999); F. Y. Liew et al, Immunology Today, Vol. 12, pp. A17-A24 (1991); B. Brune et al., Proc. Euroconference on Nitric Oxide: Basic Research and Clinical Application, Apr. 26-28, 1998; F. Stefano et al., Proc. Euroconference on Nitric Oxide: Basic Research and Clinical Application, Apr. 26-28, 1998; H. Maeda et al., J Leukocyte Biology, Vol. 56, pp. 588-592 (1994); S. Moncada, Proc. Euroconference on Nitric Oxide; Basic Research and Clinical Application, Apr. 26-28, 1988; C. G. Frostell, Proc. Euroconference on Nitric Oxide; Basic Research and Clinical Application, Apr. 26-28, 1988; G. Letts et al., Proc. Euroconference on Nitric Oxide; Basic Research and Clinical Application, Apr. 26-28, 1988; and L. C. Green et al., Analytical Biochemistry, Vol. 126, pp. 131-138 (1982), which are hereby incorporated by reference.

In some embodiments, treating a subject having a NO-related condition includes recognizing that a subject has a NO-related condition, and then administering to the subject a therapeutically effective amount of nanocrystalline silver-containing material. In certain embodiments, prophylactically treating a subject for a NO-related condition includes recognizing a subject susceptible to contracting a NO-related condition, and prophylactically treating the subject for the NO-related condition. In some embodiments, the methods can include both treating a subject having a NO-related condition and prophylactically treating a subject for a NO-related condition. For example, the methods can include treating a subject for one or more NO-related conditions and prophylactically treating the subject for one or more different NO-related conditions.

In general, a subject can be screened for a given NO-related condition using standard techniques. As an example, a subject can be screened for hypertension using a sphygnomannometer. As another example, a subject can be screened for erectile dysfunction based on reviewing the subject's medical history or using the Dic test. As a further example, a subject can be screened for angina pectoris based on reviewing the subject's medical history and/or by performing a stress EKG.

In certain embodiments, an appropriate amount of one or more additional nitrogen-containing species is administered to the subject in addition to the nanocrystalline silver-containing material. For example, if the nitrogen-containing species is in the form of nitrate ions, the nitrate ions are administered to the subject in addition to the nanocrystalline silver-containing material. In certain embodiments, the nitrogen-containing species and the nanocrystalline silver-containing material can be administered to the subject in the same solution (e.g., by injection using a needle). In some embodiments, the nitrogen-containing species and the nanocrystalline silver-containing material can be separately administered to the subject. As an example, two solutions can be administered to the subject, where one solution contains the nitrogen-containing species, and the other solution contains the nanocrystalline silver-containing material. As another example, a powder of nanocrystalline silver-containing material can be administered to the subject (e.g., using a needleless injector, using a dry powder inhaler, by ingestion), and a solution containing the nitrogen-containing species can be administered to the subject. As a further example, a pharmaceutical compositions including a nanocrystalline silver-containing material and one or more pharmaceutically acceptable carriers can be topically applied to a subject. As an additional example, a nanocrystalline silver-containing material can be ingested by a subject. As a further example, a nanocrystalline silver-containing material can be administered sublingually. As yet another example, a nanocrystalline silver-containing material can be administered to a subject using a transdermal patch. Optionally, combinations of methods/forms of administration of nanocrystalline silver-containing material can be used.

In embodiments in which the nitrogen-containing species is supplied by the nanocrystalline silver-containing material, the methods can be performed without administering to the subject a separate nitrogen-containing species, such as nitrate ions. Optionally, however, a separate nitrogen-containing species may be administered to the subject. As an example, if the nanocrystalline silver-containing material contains an appropriate amount of nitrogen, it is possible that the subject can be treated without administering a nitrogen-containing material in addition to the nanocrystalline silver-containing material.

In some embodiments, the nanocrystalline silver-containing material is administered to the subject in the form of a solution. In such embodiments, the solution can be administered to a subject with or without the use of a device. As an example, the solution can be contacted with the skin, mouth, ears or eyes as a rinse, a bath, a wash, a gargle, and/or drops. As another example, the solution can be injected using a small needle injector and/or a needleless injector. As an additional example, the solution can be formed into an aerosol (e.g., an aerosol prepared by a mechanical mister, such as a spray bottle or a nebulizer), and the aerosol can be contacted with the subject using an appropriate device (e.g., a hand held inhaler, a mechanical mister, a spray bottle, a nebulizer, an oxygen tent). As a further example, the solution can be contacted with the second location via a catheter.

In certain embodiments, the nanocrystalline silver-containing material is administered to the subject in a non-solution form. In such embodiments, the aqueous solution referred to above can be formed in the subject (e.g., by dissolution or partial dissolution of the nanocrystalline-silver containing material in body fluid within the subject). As an example, the nanocrystalline silver-containing material can be administered to the subject in the form of a powder. The powder can be, for example, inhaled (e.g., using a dry powder inhaled), ingested and/or applied topically.

In some embodiments, the NO-containing complex is administered directly to a subject. As an example, in embodiments in which a separate nitrogen-containing species is reacted with the nanocrystalline silver-containing material to for the NO-containing complex, the nanocrystalline silver-containing material and the nitrogen-containing species can be combined in an aqueous solution to form the NO-containing complex, which can then be administered to the subject. As another example, in embodiments in which the nitrogen-containing species is provided by the nanocrystalline silver-containing material, the NO-containing complex can be formed in a solution by contacting the nanocrystalline silver-containing material with an aqueous solution.

In certain embodiments, the NO-containing complex is formed within the subject. As an example, in embodiments in which a separate nitrogen-containing species is reacted with the nanocrystalline silver-containing material to for the NO-containing complex, the nanocrystalline silver-containing material and the nitrogen-containing species can be separately administered to the subject.

While certain embodiments have been described, others are also possible.

As an example, while embodiments have been described in which nanocrystalline silver-containing material is used, in some embodiments, a non-nanocrystalline material can be used that is an atomically disordered, crystalline silver-containing material.

As another example, while embodiments have been described in which nanocrystalline silver-containing materials and/or atomically disordered, crystalline silver-containing materials have been described, other such metal-containing materials can be used. Examples of nanocrystalline metal-containing materials include nanocrystalline gold-containing materials, nanocrystalline platinum-containing materials, nanocrystalline palladium-containing materials, nanocrystalline iridium-containing materials, nanocrystalline zinc-containing materials, nanocrystalline copper-containing materials, nanocrystalline tin-containing materials, nanocrystalline antimony-containing materials, and nanocrystalline bismuth containing materials. Examples of atomically disordered, crystalline metal-containing materials include atomically disordered, crystalline gold-containing materials, atomically disordered, crystalline platinum-containing materials, atomically disordered, crystalline palladium-containing materials, atomically disordered, crystalline iridium-containing materials, atomically disordered, crystalline zinc-containing materials, atomically disordered, crystalline copper-containing materials, atomically disordered, crystalline tin-containing materials, atomically disordered, crystalline antimony-containing materials, and atomically disordered, crystalline bismuth containing materials. In some embodiments, a metal-containing material is both a nanocrystalline metal-containing material and an atomically disordered, crystalline material. Such materials are disclosed, for example, in U.S. patent application Ser. No. 10/770,132, filed on Feb. 2, 2004, which is hereby incorporated by reference.

The following examples are illustrative and not intended as limiting.

### EXAMPLES

### Example 1

An adult male having hypertension is treated as follows. Silcryst®-coated Acticoat® dressing is disposed in saline solution for 30 minutes. 50 milliliters of the resulting solution are orally administered to the subject. This is repeated fours times a day for at least six months.

### Example 2

An adult male having hypertension is treated as follows. Silcryst®-coated Acticoat® dressing is disposed in saline solution for 30 minutes. 50 milliliters of the resulting solution are continually administered by IV to the subject for at least seven days.

### Example 3

An adult male having hypertension is treated as follows. A composition containing five weight percent nanocrystalline silver and a pharmaceutically acceptable carrier is topically applied to the subject. This is repeated two times a day for at least six months.

### Example 4

An adult male having erectile dysfunction is treated as follows. Silcryst®-coated Acticoat® dressing is disposed in saline solution for 30 minutes. 50 milliliters of the resulting solution are orally administered to the subject in advance of a desired erection.

### Example 5

An adult male having erectile dysfunction is treated as follows. A composition containing five weight percent nanocrystalline silver and a pharmaceutically acceptable carrier is topically applied to the subject in advance of a desired erection.

### Example 6

An adult male having erectile dysfunction is treated as follows. A composition containing five weight percent nanocrystalline silver and a pharmaceutically acceptable carrier is formed into a transdermal patch. The patch is applied to the skin of the subject for three hours in advance of a desired erection.

### Example 7

An adult male having angina pectoris is treated as described in Example 1.

### Example 8

An adult male having angina pectoris is treated as described in Example 3.

### Example 9

An adult mail having angina pectoris is treated as follows. A capsule containing two weight percent nanocrystalline silver is administered to the subject sublingually. This is repeated once a day for four weeks.

### Example 10

This example shows how nitrogen can be incorporated into sputtered nanocrystalline silver-containing materials during the sputtering process.

Samples of nanocrystalline silver powder containing differing amounts of nitrogen were produced by reactive sputtering onto a silicon wafer using a pure silver target and an argon/oxygen (5 volume % oxygen in argon) sputtering atmosphere at a pressure of about 40 mTorr, with an argon to oxygen ratio of about 10:1 and differing amounts of molecular nitrogen gas contained in the gaseous atmosphere. The temperature of the substrate remained below 50° C. under a sputtering electrical current of 0.86 Amperes for 40 minutes. The distance from target to substrate was 10 centimeters. The angle of incidence of the depositing sputtered silver flux was approximately isotropic. Following deposition the powder was scraped off the silicon wafer using a razor blade for analysis.

The amount of nitrogen contained in the samples was determined by combustion analysis (Leco analysis). The nitrogen concentration in a commercial silver oxide powder sample obtained from Sigma Chemical Company was measured to be 0.015, consistent with the nitrogen content of the sputtered sample with no molecular nitrogen gas added to the sputtering atmosphere.

Table 1 provides the amount of molecular nitrogen gas added to the sputtering atmosphere and the resulting nitrogen concentration expressed in weight percent contained in the deposited nanocrystalline material.

TABLE 1

| Amount of Molecular | Nitrogen concentration in |
|---|---|
| Nitrogen Gas in Sputtering | Deposited Nanocrystalline |
| Atmosphere (Volume Percent) | Material (Weight Percent) |
| 0 | 0.014 |
| 0.1 | 0.021 |
| 1.0 | 0.054 |
| 10.0 | 0.10 |

### Example 11

This example shows how sputtered nanocrystalline silver-containing films can react with gaseous molecular nitrogen to incorporate nitrogen.

Samples of nanocrystalline silver were reactively sputtered by sputtering onto circular 0.785 cm diameter quartz microbalance substrates using a pure silver target and an argon/oxygen sputtering atmosphere at a pressure of about 40 mTorr. The deposition was done in two stages. In the first stage, 236 milligrams were deposited on the substrates using a sputtering current of 1.2 amps for 8 minutes with a sputtering atmosphere of 6 percent oxygen in argon. In the second stage, an additional thin top coat of about 6 micrograms were deposited using a current of 0.2 amps for 2 minutes with a sputtering atmosphere of 5 percent oxygen in argon. The substrate temperature remained below 30° C. The argon to oxygen ratio was 16:1 for the first stage of deposition and about 19:1 for the second stage of deposition. The distance between the target and the substrate was 10 centimeters and the angle of incidence of the depositing sputtered silver flux was approximately isotropic.

The deposited samples were then exposed to a gaseous molecular nitrogen atmosphere at a pressure of 250 Torr and the weight increase in the samples was monitored using a quartz microbalance. Table 2 shows the data. The weight of the samples increased linearly with exposure time until reaching 0.048 micrograms (corresponding to a fractional sample weight increase of 0.02 percent after 15 hours), at which point the measurement was terminated. This indicates that the nitrogen uptake of the samples under these conditions may be of a similar magnitude to or somewhat less than that obtained by adding nitrogen to the sputtering atmosphere.

TABLE 2

| Time (hours) | Mass Gain (Micrograms) |
|---|---|
| 0 | 0 |
| 1.7 | 0.1 |
| 3.3 | 0.15 |
| 7.5 | 0.23 |
| 10.8 | 0.30 |
| 15 | 0.48 |

### Example 12

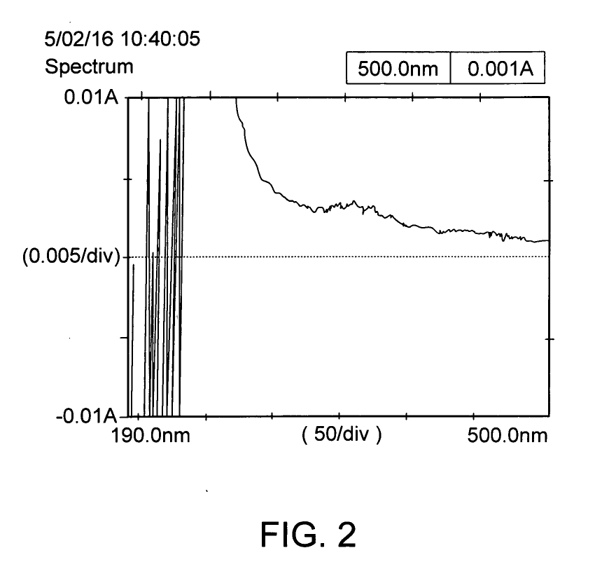

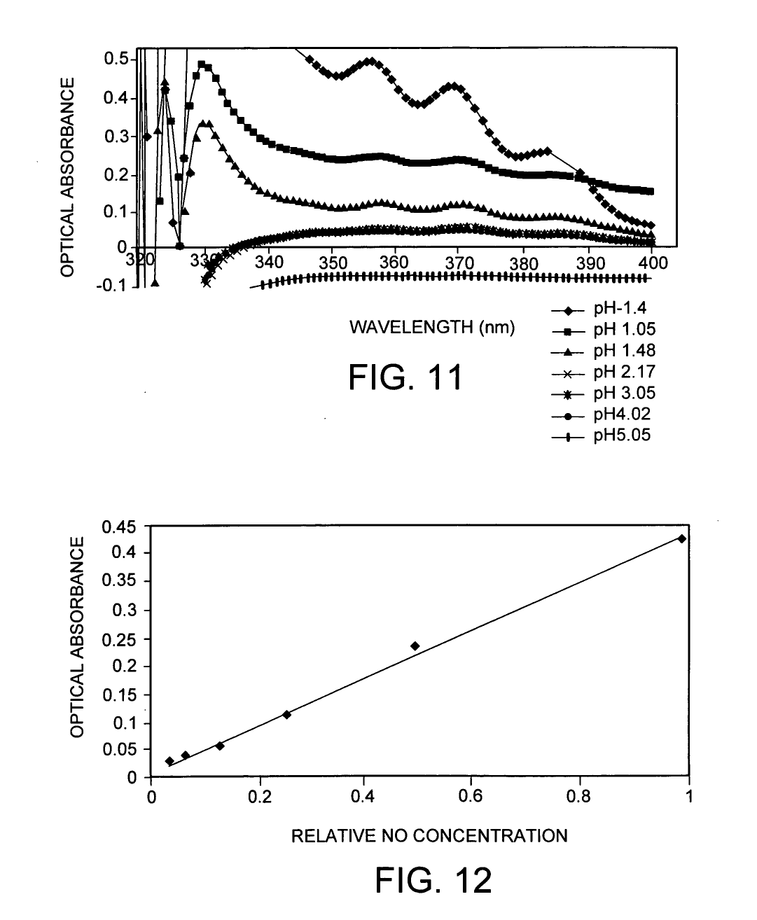

This example shows how nitric oxide can be introduced into solution from nitrogen infused nanocrystalline silver-containing materials. Samples of nanocrystalline silver-containing material having 0.1 weight percent nitrogen were prepared using the method described in Example 10. The samples were dissolved in 0.16, 0.33 and 0.66 Molar solutions of lactic acid in water. The resulting solutions were put into a quartz cuvette and their uv/visible optical absorption spectra were measured. The optical absorption spectrum for the 0.33M solution is shown in FIG. 2. This figure shows the characteristic absorption spectrum for nitric oxide with a maximum absorbance of about 0.003. The spectral lines are broadened in a manner similar to those for the spectrum in FIG. 11 corresponding to a solution of nanocrystalline silver-containing material in nitric acid adjusted to a pH of 2.17 (see discussion below). The pH of the lactic acid solution was 2.1. The remaining solutions also exhibited the characteristic absorption spectrum for nitric oxide.

### Example 13

This example confirms that the nitric oxide observed in the solutions of lactic acid described in Example 12 arose from the nitrogen infused nanocrystalline silver-containing material, by showing that materials with substantially lower nitrogen content do not produce significant concentrations of nitric oxide in solution.

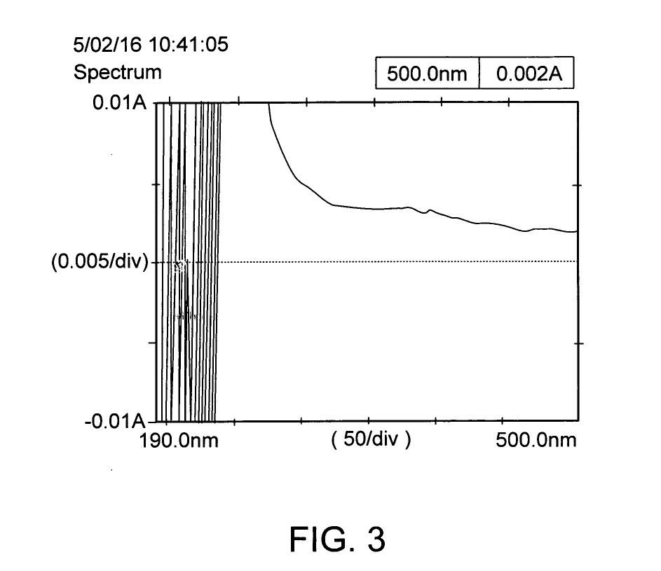

Samples of nanocrystalline silver-containing material having 0.01 weight percent nitrogen were prepared as described according to the method of Example 10. The samples were dissolved in 0.16, 0.33 and 0.66 Molar solutions of lactic acid in water as described in Example 12. The optical absorption spectra of these solutions were measured and found not to exhibit the absorption spectra for nitric oxide. The optical absorption spectrum for the 0.33M lactic acid solution is shown in FIG. 3. Assuming that the nitric oxide produced from the nanocrystalline materials is approximately proportional to their the amount of nitrogen contained in the nanocrystalline materials, the nitric oxide generated in these solutions would have been predicted to produce an optical absorbance maximum of about 0.0003, below the detection limit for the optical absorption spectrometer.

### ADDITIONAL EXAMPLES

Silver-coated Acticoat® films (Smith & Nephew, Hull, UK) including nanocrystalline silver-containing material sputtered onto high-density polyethylene net were used as a silver source for these experiments. The silver loading on the net was about 0.5 mg/cm², corresponding to a film thickness on the net of about 0.9 micrometers. The remaining reagents used were concentrated (68 to 70% assay) nitric acid obtained from Anachemia, calibrated 1N sodium hydroxide solution from Anachemia for pH adjustment and reverse osmosis grade water. 9.87% Nitric oxide gas in Nitrogen was obtained from Praxair.

The dissolution experiments were carried out in a fume hood. The silver-coated net was submerged in nitric acid diluted to 0.08N and left with or without stirring for dissolution in a 125 ml flask sealed with a rubber stopper. Stirring was done using a Corning PC-351 magnetic stirrer and a 5 centimeter stir bar. Prior to the silver dissolution, the nitric acid solution was purged with gaseous molecular nitrogen to remove dissolved oxygen. During the initial few minutes of dissolution a slow purge of gaseous molecular nitrogen at a flow rate of 1 L/min was maintained to eliminate oxygen in the headspace above the liquid to prevent re-dissolution of oxygen. The gaseous molecular nitrogen flow was then stopped and the beaker sealed to prevent stripping of nitric oxide from the solution. Dissolution was observed to proceed for several hours. The silver concentration in the solutions was monitored by extracting samples using a septum and a syringe and analyzing for silver by the atomic absorption method using Westaim Biomedical procedure WB-10-02-14 Rev 10. Concurrently, samples of solution of approximate 5 ml volume were extracted using a syringe and optical absorption spectra were obtained for the samples using Shimadzu model UV 1601 photospectrometer and quartz cuvettes with a one-centimeter optical path length. Spectra could be obtained over the wavelength range 190 to 1100 nanometers.

As a control experiment, a gas mixture of 10% nitric oxide in nitrogen was bubbled through nitric acid solution in a closed beaker that was prepurged with nitrogen. Optical absorption spectra were obtained for the resulting solution containing dissolved nitric oxide. To determine the relationship between the magnitude of the observed optical absorption peaks and the nitric oxide concentration in solution, the nitric oxide gas mixture was bubbled into a concentrated acetic acid solution. Optical absorption spectra were obtained at time intervals while the acetic acid was being saturated with nitric oxide and the concentration of nitric oxide at these times was determined by titrating the gas infused acid to reduce the nitric oxide to nitrate. The titration was performed by adding 5 milliliters of solution to a potassium iodide solution of sufficient concentration to provide excess iodide ion for the titration. The resulting solution was allowed to stand for about 30 minutes, after which 5 milliliters of a 6 gram per liter starch solution was added as a titration indicator. Following this the titration was carried out using a 0.1 normal sodium thiosulphate solution. The titration was performed slowly to accommodate the relatively slow reaction rate for the titration reaction. The optical absorbance at 371 nanometers was plotted versus the nitric oxide concentration to obtain a curve by which the nitric oxide concentration in other solutions could be determined by measuring the magnitude of the optical absorption peaks.

The solutions obtained by dissolving nanocrystalline silver-containing material were pH adjusted by taking a full sealed beaker of silver solution, removing half of the solution using a syringe and adding an equivalent volume of sodium hydroxide solution, the concentration of which was calculated to adjust the pH of the silver solution by about one pH point. Both the silver concentration and the optical absorption spectrum of each pH adjusted solution were measured prior to further pH adjustment done by repeating the process described above. Solutions with a pH up to about 7.0 were obtained and analyzed.

Additional experiments were done to determine the nature of the species created in the pH adjusted solutions. The solutions were subject to ultrafiltration using 100 and 1000 Dalton dialysis membranes. Approximately 10 ml of the solutions were placed in a pouch formed by clamping at each end a section of a tube of Spectra/Por from SPECTRUM dialysis membranes. The pouches were immersed in a 1-liter container of reverse osmosis grade water for 24 hours at ambient temperature. The membrane material was preconditioned by soaking it in reverse osmosis grade water for 1 hour prior to use. During the dialysis process the water in the container was stirred to assist the dialysis process. The solution was then removed from the pouch and added to a quartz cuvette and its optical absorption spectra was measured. The optical absorption spectrum of a control sample of solution that was not dialyzed was obtained as well, and the difference between the two spectra was calculated to show the absorption due to species removed from the solution during dialysis.

To determine whether the changes in the solutions were due to species passing through the membrane pores or to a reaction of the species with the membrane surface, a tube of 100 Dalton membrane was placed inside a larger non-permeable tube containing the same solution. The solution was kept in the reservoirs for a time equal to the dialysis time for the previous experiments, the solution was removed from the inner reservoir and its optical absorption spectrum was measured and compared with that of the undialyzed solution.

Further experiments were done to establish whether or not nitric oxide related species form when Acticoat is dissolved in water or in 0.9% saline solution. In this case 12.7 square centimeters of the silver coated high density polypropylene web used to make Acticoat was dissolved in 25 milliliters of reverse osmosis grade water or saline solution. The dissolution was carried out in a 125 milliliter beaker sealed with a rubber stopper for 18 hours in the case of the water dissolution and for 427 hours in the case of the saline solution. The differential optical absorption spectra of the solutions were measured. Finally, to look for nitrate or other nitrogen-containing species that may act as source materials for the production of nitric oxide in the presence of dissolving Acticoat, the reverse osmosis water used in the experiments was analyzed using an optical absorption method and the silver film deposited on high density polypropylene was analyzed for nitrogen using a Leco analyzer.

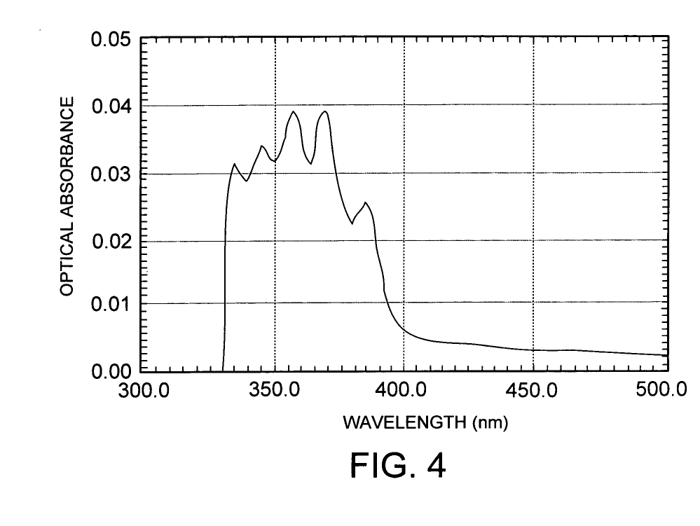

In an initial series of experiments, the main focus was on understanding the behavior of the observed optical absorption spectra for solutions containing dissolved Acticoat. A typical spectrum is shown in FIG. 4.

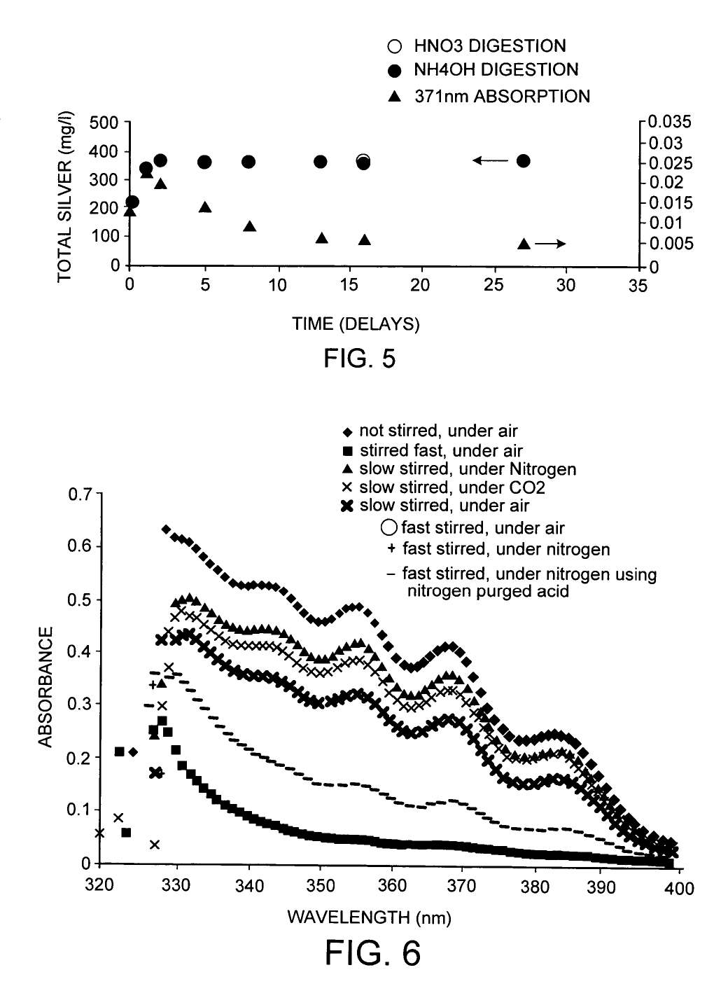

Note that there are five approximately equally spaced absorption lines between 300 and 400 nm. In an attempt to understand the origin of the spectrum, Acticoat was dissolved in a series of solutions under different conditions and the spectra were recorded at timed intervals. Some solutions were prepared by dissolving the Acticoat in an unstirred solution, and others were dissolved in solutions stirred at fast and slow rates. The slow stirring was done with the control setting on the magnetic stirrer set at 2 and fast stirring was done at the maximum setting. Additional solutions were done under a gaseous molecular nitrogen or carbon dioxide blanket, and still others were done under a gaseous molecular nitrogen blanket with the solution pre-purged with gaseous molecular nitrogen to remove dissolved gas. The height of the 371 nm peak in the absorption spectra along with the solution silver concentration for an unstirred solution as a function of dissolving time is shown in FIG. 5.

As can be seen from FIG. 5, the silver concentration initially rises rapidly, and then approaches a constant value as all of the silver is dissolved from the Acticoat. The height of the 371 nm absorption peak increases with the silver concentration initially, but then begins to decrease, indicating decay or loss of the species responsible for the peak. The remaining four absorption peaks also decreased in proportion to the 371 nm peak, indicating that all of the peaks arise from the same species in solution.

FIG. 6 shows the peak height after 30 minutes for different dissolving conditions. The spectra with the highest peaks are seen for solutions that were not stirred. The spectra with the next three highest peaks are observed for solutions that were slowly stirred, with the highest of the three for solutions under a gaseous molecular nitrogen blanket and the lowest for solutions exposed to air. The third solution in this group, which was under a carbon dioxide blanket behaved similarly to the one under nitrogen.

The four spectra for solutions that were fast stirred show the lowest peak heights. Of these the highest was for a solution initially purged with gaseous molecular nitrogen before dissolution of the Acticoat, and the others, for solutions that were not initially purged all show very low peak heights.

The results above suggest that the species responsible for the absorption peaks is volatile, since its concentration is highly dependent on the stirring speed. Since there is some dependence on the blanketing atmosphere, and on whether or not the solution was pre-purged, a secondary mechanism for dissipation of this species is reaction with dissolved gas in the solution, primarily oxygen.

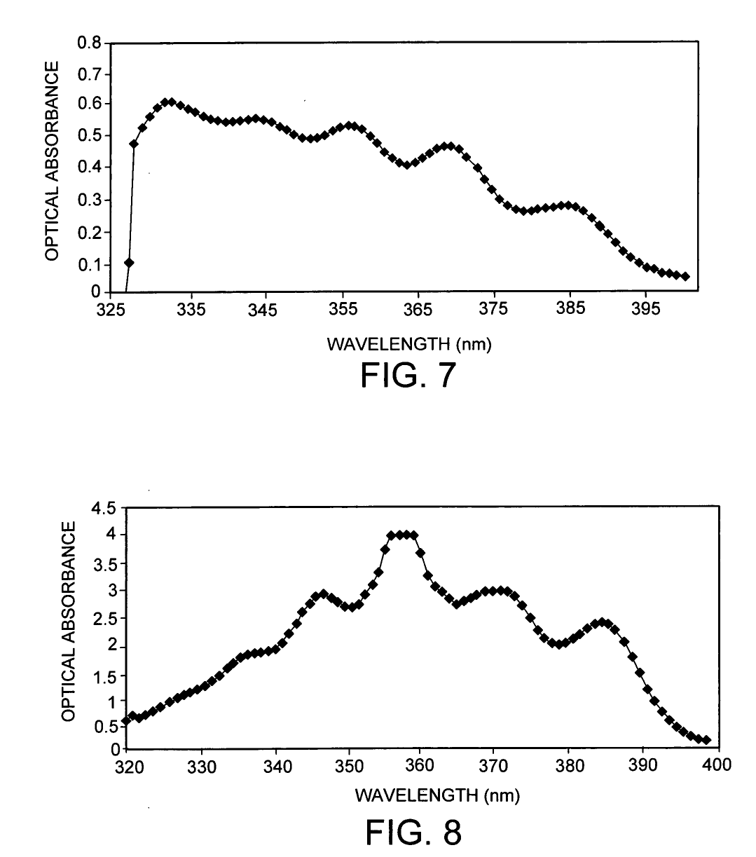

A second set of experiments were carried out to identify the species responsible for the optical absorption peaks. Based on a literature reference that relatively noble metals such as copper would react with nitrate solutions to produce nitrogen dioxide, with nitric oxide as an assumed intermediate reaction product, copper wire was dissolved in the nitric acid solution used for Acticoat dissolution and the optical absorption spectrum of the resulting solution was measured. The spectrum in the 300 to 400 nm range, shown in FIG. 7, appears identical to the one observed for the solutions of Acticoat in nitric acid, except that the peak heights are much greater.

Since no spectra for nitric oxide in aqueous solution could be found in the published literature, nitric oxide in gaseous molecular nitrogen was obtained and bubbled into nitric acid purged with gaseous molecular nitrogen to remove any dissolved molecular oxygen in the acid. The spectrum of this solution, shown in FIG. 8, also yielded the identical spectrum in the 300 to 400 nm range, so this was considered conclusive evidence that the species created by dissolution of Acticoat in nitric acid was in fact nitric oxide, and not nitrogen dioxide.

An additional set of experiments was designed to determine the concentration of nitric oxide in a solution giving rise to optical absorption peaks of a defined height. This was done by taking a solution of nitric oxide in acetic acid and titrating the nitric oxide to nitrate. The same titration method was used as described previously. This was done for several successive dilutions of the starting solution and the peak height of the 371 nm absorption line was plotted against the nitric oxide concentration determined by titration to generate a calibration curve for nitric oxide concentration measured using the height of the optical absorption peak. The calibration curve is shown in FIG. 9.

Yet another set of experiments was designed to assess the behavior of the nitric oxide in solutions containing dissolved Acticoat when the solution pH was raised towards a range that is biologically relevant. In this case, a calibrated sodium hydroxide solution was used to raise the pH in a controlled way. One requirement was to minimize transient and local excursions of the pH to very high values at which the nitric oxide would be unstable. This was accomplished by rapid stirring of the silver-containing solution while the sodium hydroxide was being added and the use of a relatively dilute sodium hydroxide solution. However, if the sodium hydroxide were to be too dilute, the silver-containing solution would be diluted excessively so the concentration of the nitric oxide would be too low for ready analysis. A compromise was reached whereby a 1 N solution of sodium hydroxide was used for the pH adjustment. However, it should be noted in these experiments that the nitrate concentration as well as the concentration of nitric oxide was being reduced as the pH was being raised. However, this occurred in a controlled and reproducible way.

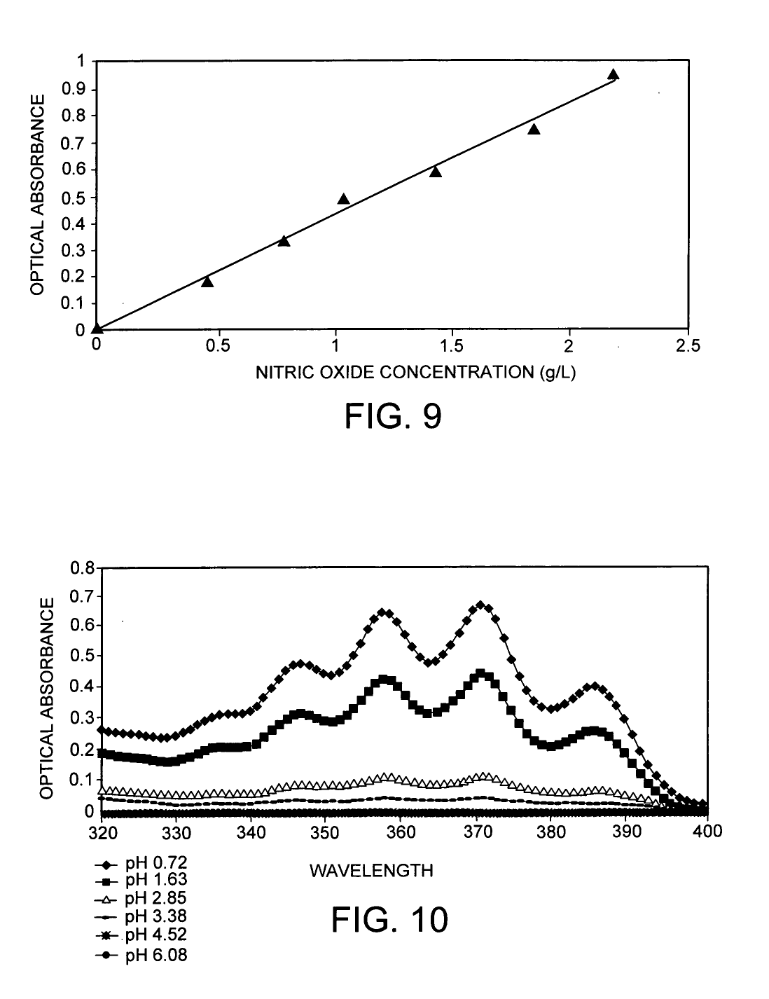

The first set of experiments to determine the effect of pH adjustment was a control set starting with nitric oxide dissolved in nitric acid. The results are shown in FIG. 10. Essentially, the shape of the spectrum remained unchanged, but the peak heights decrease in inverse proportion to the dilution factor, indicating that no appreciable chemical reaction occurred in the time frame of the experiment that involved nitric oxide. A second set of experiments was done starting with gaseous molecular nitrogen purged nitric acid in which Acticoat has been dissolved under a static gaseous molecular nitrogen blanket. The void space above the solution was minimized to limit evaporation of nitric oxide from the solution.

The results were significantly different than for nitric oxide dissolved in nitric acid. A series of spectra obtained for solutions with different pH values that were prepared as described above are plotted in FIG. 11.

As can be seen from FIG. 11, and in contrast to the results for nitric oxide dissolved in nitric acid, the peaks begin to broaden as the pH is raised, indicating an interaction between the dissolved silver species and the nitric oxide. The dependence of the peak height, independent of linewidth, is shown in FIG. 12 as a function of relative nitric oxide concentration. The relative nitric oxide concentration is calculated from the dilution factor with the assumption that the nitric oxide does not react with or volatilize from the solution.

Note that down to the lowest concentration at which the peak height could still be measured, corresponding to the solution with a pH of 4.02 and that has a relative nitric oxide concentration of 0.04, the optical absorbance bears a linear relationship with the nitric oxide concentration, indicating that the nitric oxide has not reacted or volatilized, apart from its association with the silver to cause the line broadening. Even at a pH of 5.05, there is still an indication of a broad absorption due to the nitric oxide, although the individual peaks cannot be resolved due to the line broadening. In the latter case the optical absorption due to nitric oxide is estimated to be 0.01. Using the calibration curve of FIG. 9, and the assumption that the broadened peaks are due to nitric oxide associated with silver, and that the molar absorptivity of the nitric oxide is unchanged in these circumstances, the concentration of nitric oxide at a pH of 5.05. is calculated to be about 20 parts per million, which is greater than the concentrations of less than 1 part per million reported in the literature as having significant biological effects. Even allowing a further shift in the pH to a neutral value typical for biological systems, it seems likely that sufficient nitric oxide would be produced by Acticoat to provide the efficacy reported for nitric oxide, provided that an adequate concentration of nitrate ion is present. Regarding the nitrate concentration in biological systems, Green et al (5) report a nitrate ion concentration in the range of 15 to 85/μM (1 to 5 parts per billion) for blood plasma and gastric juice. This appears insufficient for the direct production of nitric oxide in a biologically significant concentration range from nitrate, although it is possible that local nitrate concentrations close to synthesis sites are much higher.

Similar experiments to those described in the preceding paragraphs were carried out by dissolving silver wire rather than Acticoat in nitric acid. It was found that the solubility of the silver wire was extremely limited. The very much greater solubility of silver from Acticoat particularly at pH values in the biologically relevant range may be due to its physical/chemical (nanocrystalline composite) nature. It is known from previous studies that Acticoat includes a soluble silver oxide phase in a metallic silver matrix phase. When it is dissolved in water, the oxide phase is dissolved in a few hours, leaving a porous silver matrix phase with an extremely high surface area, allowing the metallic phase to dissolve at a slower rate, possibly in conjunction with oxygen dissolved in the water. This slow, but measurable rate of silver dissolution may facilitate the generation of enough nitric oxide from the small but finite concentration of nitric oxide in biological systems to create a dynamic balance of nitric oxide in solution. The concentration in dynamic equilibrium would be determined by the relative rate of production and consumption of nitric oxide due to oxidation and other biological processes.

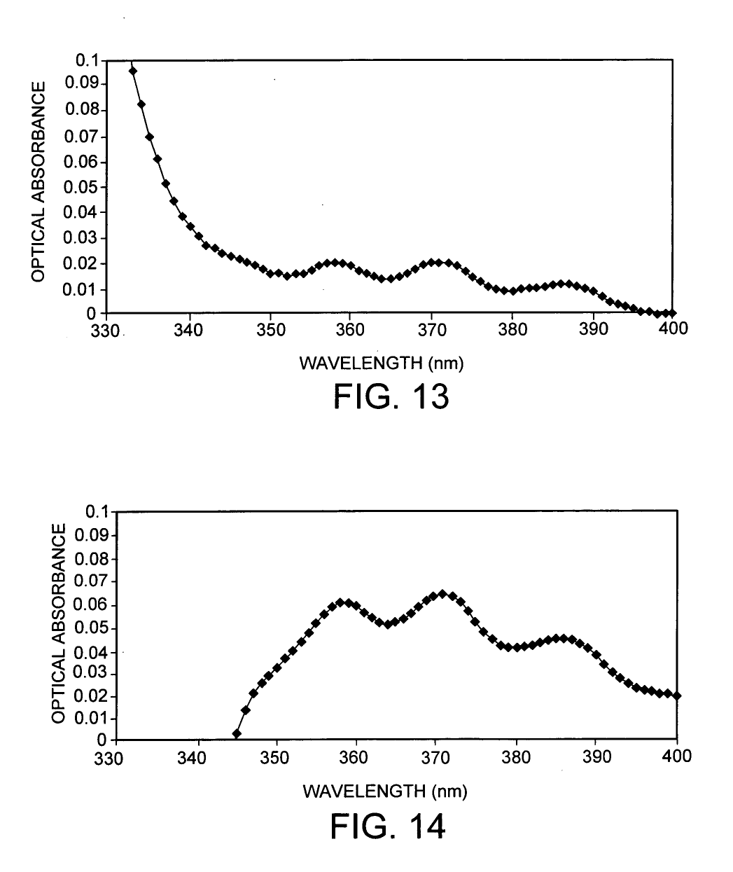

To determine the approximate size of the species responsible for the broadened optical absorption peaks in pH adjusted solutions of Acticoat, a solution adjusted to a pH of 3.38 was subject to dialysis in a 100 Dalton membrane pouch. The optical absorption spectrum before dialysis with the spectrum after dialysis subtracted from it is shown in FIG. 13.

For comparison the absorption spectrum prior to dialysis with the spectrum for water subtracted from it is shown in FIG. 14. The features in the spectrum of FIG. 13 correspond to species removed from the solution by dialysis. As can be seen from this figure, the broad feature centered at 360 nanometers in FIG. 13 is reduced in amplitude by about 70% as compared to the same feature in the undialyzed solution following dialysis using the 100 Dalton membrane. This indicates that about 30% of the concentration of species responsible for the feature, namely the free and adsorbed nitric oxide species are able to pass to some extent through the 100 Dalton pores. Thus the dimension of the species is in the range of about 50 Angstroms.

Note that for this solution the spectral feature consists of the relatively narrow nitric oxide peaks superimposed on a broad absorption centered at 360 nanometers. The increasing presence of the broad absorption with higher pH was inferred from the observation that the ratio of the absorbance at the center of the narrow peaks divided by the absorbance minimum between the peaks is significantly closer to unity than for spectra obtained on solutions with a lower pH. Both the narrow lines and the broad absorption species are removed to about the same extent from the dialyzed solution. The reduction is expected for the narrow lines, since they are due to free nitric oxide in the solution, but the result also shows that the species responsible for the broad absorption are small enough to pass through the membrane.

To determine whether or not species removed from the dialyzed solutions actually passed through the membrane pores, or were adsorbed onto or reacted with the membrane surface, an experiment was run with pH adjusted solution on both sides of a 100 Dalton membrane as described earlier in this report. The optical absorption spectra of the solution was measured following storage of the solution in the presence of the membrane and compared to the spectrum for the initial solution. The spectra were very similar, indicating that very little reaction occurred at the surface of the membrane and that the species removed on the dialysis experiments did in fact pass through the membrane pores.

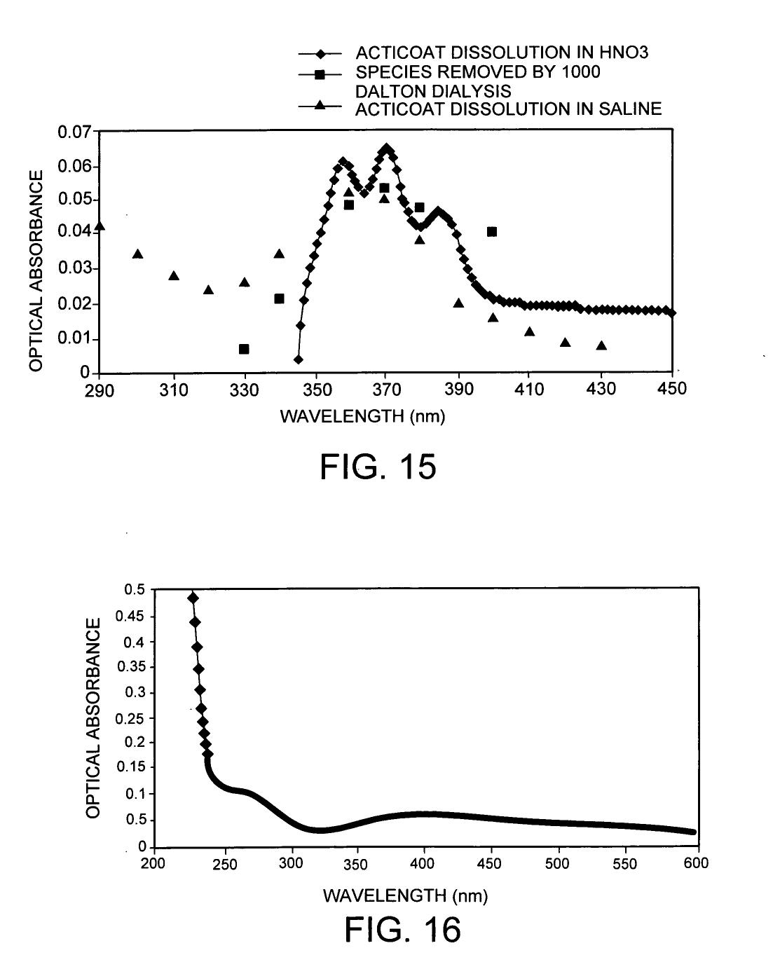

Dialysis experiments using a 1000 Dalton membranes were also conducted on a pH adjusted solution having a pH of 4. FIG. 15 shows the difference between the optical absorption spectra for dialyzed and undialyzed solutions, as well as the spectrum for the undialyzed solution. As with the 100 Dalton membrane, the difference spectrum shows some removal by dialysis of the broad absorption feature centered at about 360 nanometers.

However, even with the significantly larger membrane pore, not all of the species responsible for the absorption in this wavelength range were removed, indicating that the complexed species in the higher pH solution may be larger than in the lower pH solution.

To correlate the results obtained by adjusting the pH of the nitrate solutions containing dissolved Acticoat with species created by dissolving Acticoat in water, Acticoat was dissolved in water for 336 hours at 37° C. under gaseous molecular nitrogen and the optical absorption spectrum was obtained as shown in FIG. 16.

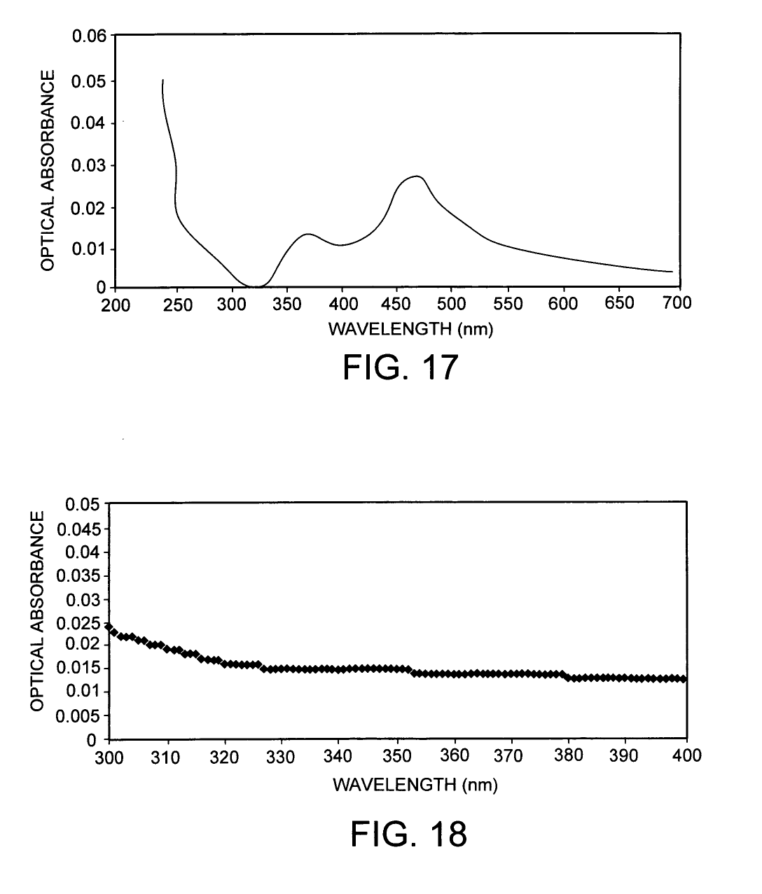

This solution was then subject to dialysis using a 1000 Dalton membrane. The difference between the optical absorption spectra is shown with a magnified absorbance scale in FIG. 17. As can be seen from the figure, the large peak below 300 nanometers that corresponds to Ag⁺ ions in FIG. 16 is substantially reduced, as is expected. The peak at about 420 nanometers is not observed, indicating that colloidal silver particles are not removed by dialysis. However, with these absorptions subtracted out in the difference spectrum, a peak is revealed centered at about 360 nanometer corresponding to a species removed by the dialysis. This peak is very similar to the broad peak found in pH adjusted solutions of Acticoat in nitric acid, suggesting that the species responsible for it may be the same in both cases. The species may include neutral silver atoms as identified as being present in solutions of Acticoat films dissolved in water in electrochemical measurement described in A. Bard et al., J. Electrochem. Soc., Vol. 106, No. 2, pp. 279-287 (2002).

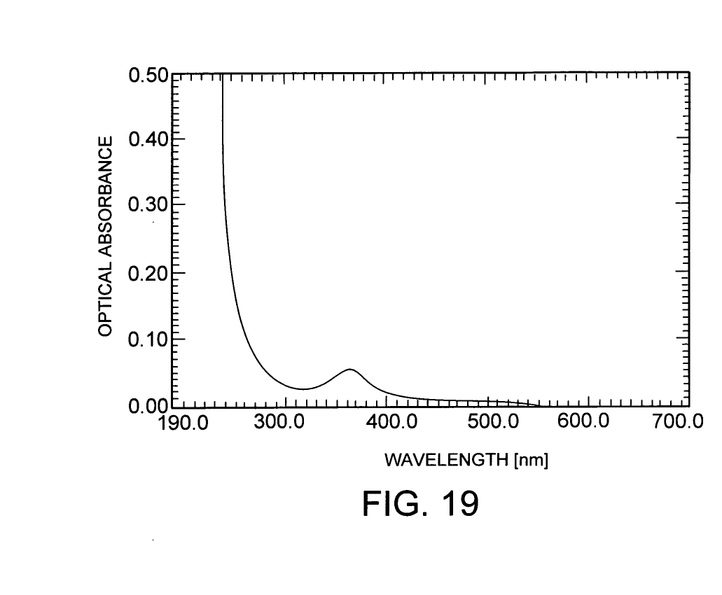

A further experiment was done to evaluate species formed by dissolving Acticoat in 0.9% saline (NaCl) solution. This salt concentration is similar to that in biological fluids. Although the optical absorption spectrum for the resulting solution shown in FIG. 18 did not show a peak at 360 nanometers after 50 hours of dissolution, the spectrum after 384 hours of dissolution shown in FIG. 19 does show the peak, again suggesting the presence of the nitric oxide containing species. The same optical absorption data is shown in FIG. 15 compared against the spectra obtained by pH adjusting solutions of Acticoat dissolved in nitric acid. The presence of these complexed silver-containing species may account for the silver content in the solutions that is much higher that the silver ion concentration predicted from the solubility of silver chloride.

Finally the nitrate concentration in the reverse osmosis water used in the experiments was analyzed and found to be <0.2 mg/L, corresponding to less than 200 parts per billion. However, the nitrogen content in the Acticoat films was measured to be 3.4% weight percent, present largely in the Delnet but also present to some degree within the silver film. This is more than sufficient, if it is in a nitrate-forming state, to react with nanocrystalline silver-containing material to generate nitric oxide.

Substantial evidence has been found for the generation of nitric oxide when Acticoat is dissolved in acidic solutions containing nitrate. The nitric oxide appears to become adsorbed onto clusters of silver atoms formed when the pH of these solutions is raised by the addition of sodium hydroxide solution. Analysis of spectra for aqueous and saline solutions that have been subject to dialysis show optical absorption features that appear to coincide with the spectrum from the adsorbed nitric oxide, indicating that nitric oxide may also form when Acticoat is dissolved in biological solutions. The remaining question is the origin of the nitrogen-containing species in these biological solutions that is reduced to form the nitric oxide, although it is known that nitrate ions are present in such solutions in concentrations of several hundred parts per billion.

Other embodiments are in the claims.

1. A method of treating a subject having a NO-related condition, the method comprising:
recognizing that the subject has the NO-related condition; and
administering to the subject a therapeutically effective amount of a nanocrystalline metal-containing material.

2. The method of claim 1, wherein the nanocrystalline metal-containing material comprises a nanocrystalline silver-containing material.

3. The method of claim 2, wherein the nanocrystalline silver-containing material is an atomically disordered, crystalline material.

4. The method of claim 1, wherein the nanocrystalline metal-containing material forms a NO-containing complex.

5. The method of claim 1, wherein, prior to being administered to the subject, the nanocrystalline metal-containing material is in contact with a solution.

6. The method of claim 5, wherein the solution comprises a body fluid.

7. The method of claim 1, wherein the nanocrystalline metal-containing material is injected into the subject, topically applied to the subject, ingested by the subject or inhaled by the subject.

8. The method of claim 1, wherein the nanocrystalline metal-containing material is administered in the form of a tape, a pill, a capsule, a tablet, a lozenge or a suppository.

9. The method of claim 1, wherein the nanocrystalline metal-containing material is administered as a powder.

10. The method of claim 1, wherein the nanocrystalline metal-containing material is administered in the form of a pharmaceutical composition that comprises the nanocrystalline metal-containing material and at least one pharmaceutically acceptable carrier.

11. The method of claim 1, wherein the nanocrystalline metal-containing material is reacted with a nitrogen-containing species to form a NO-containing complex.

12. A method of treating a subject having a NO-related condition, the method comprising:
recognizing that the subject has a NO-related condition; and
administering to the subject a therapeutically effective amount of a source of solution phase neutral silver.

13. The method of claim 12, further comprising reacting the solution phase neutral silver with a nitrogen-containing species to form NO.

14. The method of claim 13, wherein the NO forms a NO-containing complex.

15. The method of claim 12, wherein the neutral silver is present in an aqueous solution.

16. A method of treating a subject having a NO-related condition, the method comprising:
recognizing that the subject has a NO-related condition; and
administering to the subject a therapeutically effective amount of a NO-containing complex.

17. The method of claim 16, wherein the NO-containing complex is in contact with a solution.

18. The method of claim 17, wherein the solution comprises a body fluid.

19. A method of treating a subject susceptible to having a NO-related condition, the method comprising:
recognizing that the subject is susceptible to having a NO-related condition; and
administering to the subject a therapeutically effective amount of a nanocrystalline metal-containing material.

20. The method of claim 19, wherein the nanocrystalline metal-containing material comprises a nanocrystalline silver-containing material.

21. The method of claim 20, wherein the nanocrystalline silver-containing material is an atomically disordered, crystalline material.

22. The method of claim 19, wherein the nanocrystalline metal-containing material forms a NO-containing complex.

23. The method of claim 19, wherein, prior to being administered to the subject, the nanocrystalline metal-containing material is in contact with a solution.

24. The method of claim 23, wherein the solution comprises a body fluid.

25. The method of claim 23, wherein the nanocrystalline metal-containing material is injected into the subject, topically applied to the subject, ingested by the subject or inhaled by the subject.

26. The method of claim 19, wherein the nanocrystalline metal-containing material is administered as a powder.

27. The method of claim 19, wherein the nanocrystalline metal-containing material is reacted with a nitrogen-containing species to form a NO-containing complex.

28. A method of treating a subject susceptible to having a NO-related condition, the method comprising:
recognizing that the subject is susceptible to having the NO-related condition; and
administering to the subject a therapeutically effective amount of a source of solution phase neutral silver.

29. The method of claim 28, further comprising reacting the solution phase neutral silver with a nitrogen-containing species to form NO.

30. The method of claim 29, wherein the NO forms a NO-containing complex.

31. The method of claim 28, wherein the neutral silver is present in an aqueous solution.

32. A method of treating a subject susceptible to having a NO-related condition, the method comprising:
recognizing that the subject is susceptible to having the NO-related condition; and
administering to the subject a therapeutically effective amount of a NO-containing complex.

33. The method of claim 32, wherein the NO-containing complex is in contact with a solution.

34. The method of claim 33, wherein the solution comprises a body fluid.

35-53. (canceled)
